(19) United States
(12) Patent Application Publication
(10) Pub. No.: US 2016/0135775 A1
(43) Pub. Date: May 19, 2016

(54) System And Method For Time-Resolved, Three-Dimensional Angiography With Physiological Information

(71) Applicant: Wisconsin Alumni Research Foundation, Madison, WI (US)

(72) Inventors: Chuck Mistretta, Madison, WI (US); Charlie Strother, Madison, WI (US)

(21) Appl. No.: 14/542,822

(22) Filed: Nov. 17, 2014

(51) Int. Cl. A61B 6/00; A61B 8/06; A61B 5/00; G06T 7/00; G06T 5/50; G06T 15/08; G06T 11/00; A61B 5/055; A61B 8/08

(52) U.S. Cl. CPC A61B 6/5247; A61B 5/055; A61B 8/06; A61B 6/481; A61B 5/0035; A61B 8/488; A61B 8/483; A61B 8/5261; G06T 5/50; G06T 15/08; G06T 11/003; G06T 7/0012; G06T 2207/10116; G06T 2211/404; G06T 2207/10136; G06T 2207/10076; G06T 2207/10088; G06T 2200/04; G06T 2207/30104

(57) ABSTRACT

A system and method are provided for generating time resolved series of angiographic volume data having velocity or velocity-derived information integrated therewith. An image processing system is configured to receive angiographic volume data and flow sensitive imaging data and process the angiographic volume data and flow sensitive imaging data to generate a combined dataset. A display is configured to display the combined dataset as angiographic volumes having associated time-resolved, color-coded flow information.

### CROSS-REFERENCE TO RELATED APPLICATIONS

N/A

### BACKGROUND

The present disclosure is directed to angiography and, in particular, the disclosure relates to a system and method for producing time-resolved, three-dimensional (3D) angiographic images including physiological information, such as flow information.

Since the introduction of angiography beginning with the direct carotid artery punctures of Moniz in 1927, there have been ongoing attempts to develop angiographic techniques that provide diagnostic images of the vasculature, while simultaneously reducing the invasiveness associated with the procedure. In the late 1970's, a technique known as digital subtraction angiography (DSA) was developed based on real-time digital processing equipment. Due to steady advancements in both hardware and software, DSA can now provide depictions of the vasculature in both 2D and rotational 3D formats. Three-dimensional digital subtraction angiography (3D-DSA) has become an important component in the diagnosis and management of people with a large variety of central nervous system vascular diseases.

In recent years competition for traditional DSA has emerged in the form of computed tomography angiography (CTA) and magnetic resonance angiography (MRA). CTA provides high spatial resolution, but is not time-resolved unless the imaging volume is severely limited. CTA is also limited as a standalone diagnostic modality by artifacts caused by bone at the skull base and the contamination of arterial images with opacified venous structures. Further, CTA provides no functionality for guiding or monitoring minimally-invasive endovascular interventions. Significant advances have been made in both the spatial and the temporal resolution qualities of MRA. Currently, gadolinium-enhanced time-resolved MRA (TRICKS) is widely viewed as a dominant clinical standard for time-resolved MRA. TRICKS enables voxel sizes of about 10 mm3 and a temporal resolution of approximately 10 seconds. Advancements such as HYBRID highly constrained projection reconstruction (HYPR) MRA techniques, which violate the Nyquist theorem by factors approaching 1000, can provide images with sub-millimeter isotropic resolution at frame times just under 1 second. Nonetheless, the spatial and temporal resolution of MRA are not adequate for all imaging situations and its costs are considerable.

The recently-introduced, four-dimensional (4D) DSA techniques can use rotational DSA C-arm imaging systems controlled with respect to a particular injection timing so that there is time dependence in the acquired projections. As described in U.S. Pat. No. 8,643,642, which is incorporated herein by reference, a 3D DSA volume can be used as a constraining volume to generate a new 3D volume that uses the temporal information in each projection. As in 3D DSA, a mask rotation without contrast is followed by a second rotation in which contrast is injected. The process can create a series of 3D angiographic volumes that can be updated, for example, every 1/30 of a second.

Thus, the above-described systems and methods have improved over time and, thereby, provided clinicians with an improving ability to visualize the anatomy of the vessels being studied. Of course, vessels are dynamic and functional structures and the specifics of the anatomy can be used by the clinician to deduce information about the dynamic and functional nature of the vessels. Put another way, with ever increasing spatial and temporal resolution, the clinician has been provided with clearer and more accurate information about the geometry of the vessel. As such, the deductions made by the clinician about the dynamics and function of the vessel have correspondingly improved. Unfortunately, even the best deductions are still inherently limited.

Therefore, it would be desirable to have a system and method for providing information about the function or dynamic performance of the anatomy to a clinician performing an angiographic study.

### SUMMARY

The present disclosure overcomes the aforementioned drawbacks by providing a system and method for integrating functional and/or dynamic performance information with high-quality anatomical angiographic images. In particular, a system and method is provided that can integrate flow information with a time-resolved angiographic study, including 4D DSA studies. In one configuration, velocity information is derived using an imaging modality, such as magnetic resonance imaging (MRI) or ultrasound, and integrated with information acquired when performing an angiographic study to provide time-resolved, anatomical angiographic images that include flow or velocity and velocity-derived information.

In accordance with one aspect of the disclosure, a system is provided for generating time resolved series of angiographic volume data having velocity or velocity-derived information integrated therewith. The system includes an image processing system configured to receive angiographic volume data and flow sensitive imaging data and process the angiographic volume data and flow sensitive imaging data to generate a combined dataset. The system also includes a display configured to display the combined dataset as angiographic volumes having associated time-resolved, color-coded flow information.

In accordance with another aspect of the disclosure, a method is provided for generating time resolved series of angiographic volume data having velocity or velocity-derived information integrated therewith. The method includes generating a series of 3D time-resolved vascular volumes from time resolved x-ray projection data and generating a series of flow-sensitive 3D volumes from one of magnetic resonance imaging data or ultrasound data. The method also includes integrating the series of 3D time-resolved vascular volumes and the series flow-sensitive 3D volumes to generate a series of time-resolved vascular volumes that have voxel intensities or color coding defined by at least one of iodine concentration derived from the time resolved x-ray projection data or velocity information derived from the one of magnetic resonance imaging data or ultrasound data.

In accordance with yet another aspect of the disclosure, a system is provided for generating time resolved series of angiographic volume data having velocity or velocity-derived information integrated therewith. The system includes a source of x-ray data of a subject having received a dose of a contrast agent and a source of at least one of magnetic resonance imaging data or ultrasound data of the subject. The system also includes a processing system having access to the source of x-ray data and the source of at least one of magnetic resonance imaging data or ultrasound data. The processing system is configured to generate a time-series of two-dimensional images from the x-ray data, each of the two-dimensional images corresponding to a different time in the time period and a different angle relative to the subject, wherein each of the two-dimensional images comprises pixel intensity information. The processing system is also configured to generate a three-dimensional image without temporal resolution from the x-ray data. The processing system is further configured to determine, for each of a plurality of the two-dimensional images, voxel weightings in the three-dimensional image without temporal resolution by multiplying the voxels with the pixel intensity information of a two-dimensional image in the plurality. The processing system is also configured to produce a time-resolved three-dimensional image of the subject by selectively combining the three-dimensional image without temporal resolution and the time-series of two-dimensional images, the voxel weightings being used to nullify one or more voxels from the three-dimensional image without temporal resolution to produce the time-resolved three-dimensional image. The processing system is additionally configured to produce a series of velocity images of the subject, wherein each of the velocity images comprises pixel color information weighted based on flow information associated derived from the at least one of magnetic resonance imaging data or ultrasound data. The processing system is also configured to combine the series of velocity images of the subject with the time-resolved three-dimensional image of the subject to generate a series of time-resolved vascular volumes that have voxel intensities or color coding defined by the flow information.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is a block diagraph of a system for creating images having integrated flow information with a time-resolved angiographic images in accordance with the present disclosure.

FIG. 1B is a schematic diagram illustrating a process that can be carried out with the system of FIG. 1A in accordance with the present disclosure.

FIGS. 2A and 2B is a perspective and block diagram, respectively, of an example of an x-ray imaging system that can be used in accordance with the present disclosure to acquire angiographic data.

FIG. 3 is a flow chart setting forth examples of steps for producing a time-resolved 3D image or 4D DSA dataset from x-ray data.

FIG. 4 is a flow chart setting forth further examples of steps for producing a time-resolved 3D image or 4D DSA dataset from x-ray data.

FIG. 5 is a graphic depiction of selection combination of a 3D image with a 2D DSA image frame to produce 4D DSA data.

FIG. 6 is a schematic block diagram of a magnetic resonance imaging (MRI) system for use in accordance with the present disclosure.

FIG. 7 is a flow chart for acquiring flow sensitive data using the MRI system of FIG. 6.

FIG. 8 is a graphic illustration of k-space sampling strategy for acquiring flow sensitive data.

FIG. 9 is a pulse sequence for use with the MRI system of FIG. 6 to acquire flow sensitive data.

FIG. 10A is another graphic illustration of k-space sampling strategy for acquiring flow sensitive data.

FIG. 10B is yet another graphic illustration of k-space sampling strategy for acquiring flow sensitive data.

FIG. 11 is a schematic diagram of a ultrasound system for use with the present invention to acquire flow sensitive data.

FIG. 12 is a block diagram of one example of steps for creating 7 dimensional (7D) DSA images in accordance with the present disclosure.

FIG. 13 is a block diagram of one example of steps for creating 7 dimensional (7D) DSA images from MIR 3D velocity data and 4D DSA data in accordance with the present disclosure.

FIG. 14 is an example of a 7D DSA image created in accordance with the present disclosure.

### DETAILED DESCRIPTION

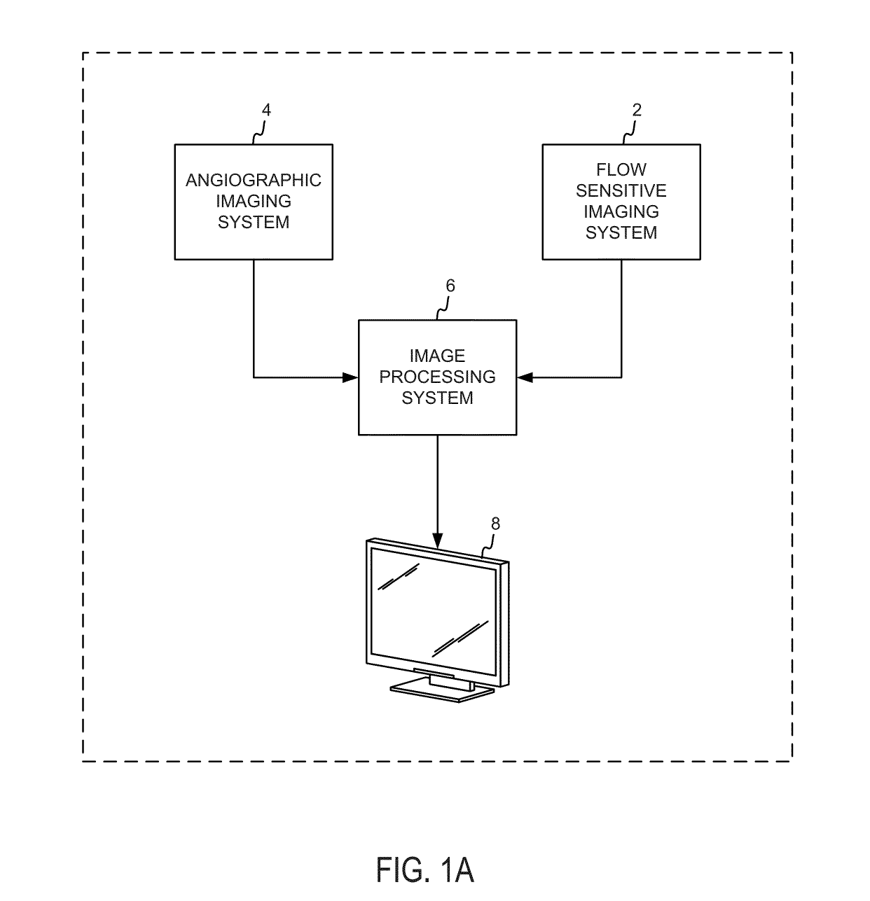

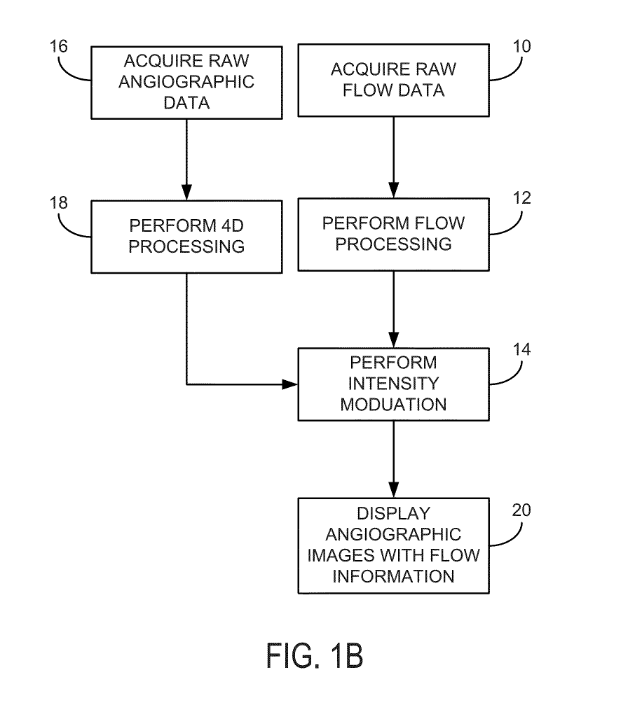

Referring to FIG. 1A, a system is illustrated for creating time-resolved angiographic images that are integrated with physiological or functional information. In particular, the system includes a flow sensitive imaging system 2 and a angiographic imaging system 4 that both provide data to an image processing system 6 to provide time-resolved angiographic images having physiological or functional information to a display 8. To this end, as will be further described in detail, clinicians, including surgeons, interventional radiologists, and the like, can be provided with time-resolved angiographic images that include flow or velocity information.

As illustrated in FIG. 1B, raw data including flow or velocity information is acquired at process block 10 and is processed to create images having flow information at process block 12. Thereafter, the images having flow information are intensity modulated at process block 14, as will be described, with angiographic images. that is, raw angiographic data may be acquired at process block 16. At process block 18, the raw angiographic data is processed to create 4D angiographic images, such as 4D DSA images. The 4D angiographic images are then integrated with the flow images at process block 14 to create images that are displayed at process block 20 as angiographic images having flow information.

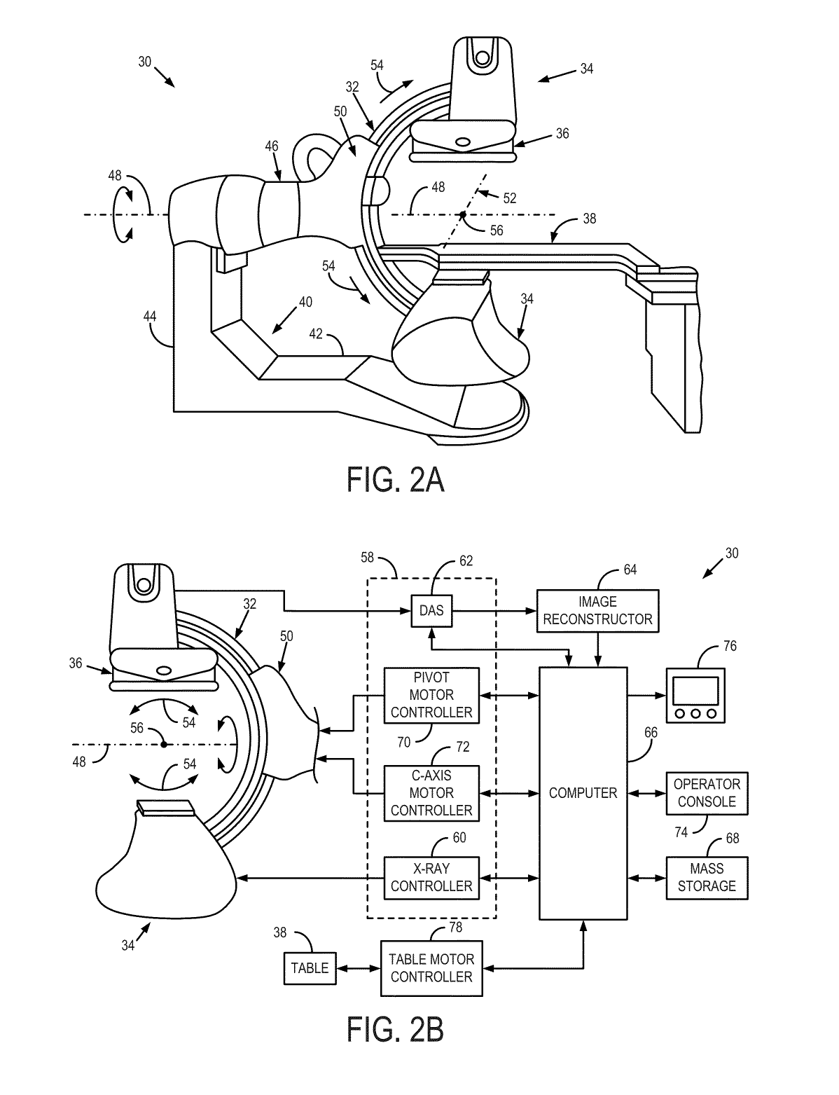

Referring now to FIGS. 2A and 2B, an example of an x-ray imaging system 30 is illustrated. The x-ray imaging system 30 is illustrated as a so-called “C-arm” imaging system; however, other geometries may be used to acquired x-ray angiographic images. For example, any of a variety of x-ray imaging systems capable of acquiring data to create a 4D-DSA image may be used, including systems that acquire time-resolved 2D images using a single plane x-ray system.

The imaging system 30, as illustrated, may be generally designed for use in connection with interventional procedures. The imaging system 30 is characterized by a gantry 32 forming a C-arm that carries an x-ray source assembly 34 on one of its ends and an x-ray detector array assembly 36 at its other end. The gantry 32 enables the x-ray source assembly 34 and detector array assembly 36 to be oriented in different positions and angles around a patient disposed on a table 38, while enabling a physician access to the patient.

The gantry includes a support base 40, which may include an L-shaped pedestal that has a horizontal leg 42 that extends beneath the table 38 and a vertical leg 44 that extends upward at the end of the horizontal leg 42 that is spaced from of the table 38. A support arm 46 is rotatably fastened to the upper end of vertical leg 44 for rotation about a horizontal pivot axis 48. The pivot axis 48 is aligned with the centerline of the table 38 and the support arm 46 extends radially outward from the pivot axis 48 to support a drive assembly 50 on its outer end. The C-arm gantry 32 is slidably fastened to the drive assembly 50 and is coupled to a drive motor (not shown) that slides the C-arm gantry 32 to revolve it about a C-axis 52, as indicated by arrows 54. The pivot axis 48 and C-axis 52 intersect each other at an isocenter 56 that is located above the table 408 and they are perpendicular to each other.

The x-ray source assembly 34 is mounted to one end of the C-arm gantry 32 and the detector array assembly 36 is mounted to its other end. As will be discussed in more detail below, the x-ray source assembly 34 includes an x-ray source (not shown) that emits a beam of x-rays, which are directed at the detector array assembly 36. Both assemblies 34 and 36 extend radially inward to the pivot axis 38 such that the center ray of this cone beam passes through the system isocenter 56. The center ray of the x-ray beam can, thus, be rotated about the system isocenter 56 around either the pivot axis 38, the C-axis 52, or both during the acquisition of x-ray attenuation data from a subject placed on the table 38.

As mentioned above, the x-ray source assembly 34 contains an x-ray source that emits a beam of x-rays when energized. The center ray passes through the system isocenter 56 and impinges on a two-dimensional flat panel digital detector housed in the detector assembly 36. Each detector element produces an electrical signal that represents the intensity of an impinging x-ray and, hence, the attenuation of the x-ray as it passes through the patient. During a scan, the x-ray source and detector array are rotated about the system isocenter 56 to acquire x-ray attenuation projection data from different angles. By way of example, the detector array is able to acquire thirty projections, or views, per second. Generally, the numbers of projections acquired per second is the limiting factor that determines how many views can be acquired for a prescribed scan path and speed. Accordingly, as will be described, this system or others can be used to acquire data that can be used to crate 4D DSA image data sets that may provide 3D angiographic volumes at the rate of, for example, 30 per second.

Referring particularly to FIG. 2B, the rotation of the assemblies 34 and 36 and the operation of the x-ray source are governed by a control system 58 of the imaging system 30. The control system 58 includes an x-ray controller 60 that provides power and timing signals to the x-ray source. A data acquisition system (DAS) 62 in the control system 58 samples data from detector elements in the detector array assembly 36 and passes the data to an image reconstructor 64. The image reconstructor 64, receives digitized x-ray data from the DAS 62 and performs image reconstruction. The image reconstructed by the image reconstructor 64 is applied as an input to a computer 66, which stores the image in a mass storage device 68 or processes the image further.

The control system 58 also includes pivot motor controller 70 and a C-axis motor controller 72. In response to motion commands from the computer 66, the motor controllers 70 and 72 provide power to motors in the imaging system 30 that produce the rotations about the pivot axis 38 and C-axis 52, respectively. A program executed by the computer 66 generates motion commands to the motor controllers 70 and 72 to move the assemblies 34 and 36 in a prescribed scan path.

The computer 66 also receives commands and scanning parameters from an operator via a console 74 that has a keyboard and other manually operable controls. An associated display 76 allows the operator to observe the reconstructed image and other data from the computer 66. The operator supplied commands are used by the computer 66 under the direction of stored programs to provide control signals and information to the DAS 62, the x-ray controller 60, and the motor controllers 70 and 72. In addition, the computer 66 operates a table motor controller 78, which controls the patient table 408 to position the patient with respect to the system isocenter 56.

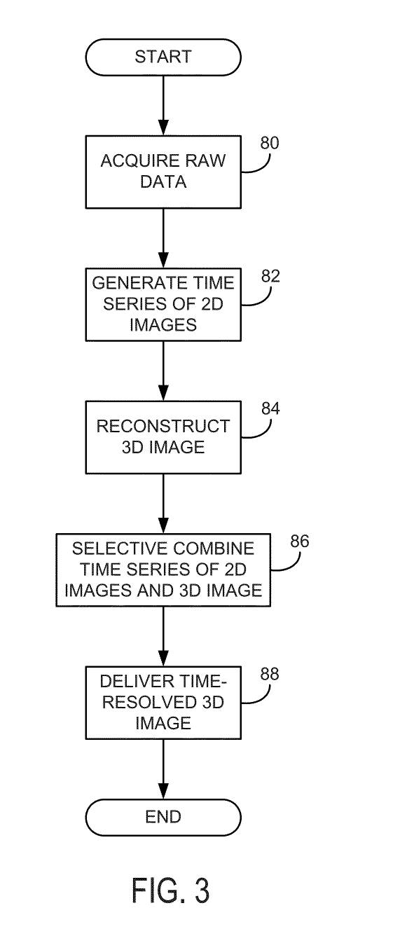

The above-described system can be used to acquire raw angiographic data that can then be processed to generate a time-resolved 3D angiographic image in the form of a 4D DSA image. Referring to FIG. 3, a process for creating a 4D DSA image begins at process block 80 with the acquisition of image data from a region-of-interest in a subject using a medical imaging system, such as a CT system or a single-plane, biplane, or rotational x-ray systems. At process block 82, a time-series of 2D images is generated from at least a portion of the acquired image data. While the time-series of 2D images can have a high temporal and spatial resolution and may include images acquired at different angles around the subject, it generally cannot provide a sophisticated 3D depiction of the subject. At process block 84, a 3D image of the subject is reconstructed from the acquired image data. Though individual projections used to reconstruct this 3D image may themselves convey some degree of temporal information, the reconstructed 3D image itself is substantially free of temporal resolution. For brevity, the 3D image substantially without temporal resolution and the time-series of 2D images may simply be referred to as the “3D image” and “2D images,” respectively.

At process block 86, the time-series of 2D images and the static 3D image are selectively combined so that the temporal information included in the 2D images is imparted into the 3D image. This results in the production of a time-resolved 3D image of the subject with high temporal and spatial resolution. While the selective combination process varies based on the medical imaging system used and the nature of the acquired image data, it generally involves the steps of (1) registering the 2D images to the 3D image, (2) projecting the attenuation value of the pixels in the 2D images into the 3D image, and (3) weighting the 3D image with the projected values for each individual frame of the time-series of 2D images. It is contemplated that the temporal weighting in step (3) generally involves multiplying the projected pixel values with the 3D image. These three steps, which can be referred to as “multiplicative projection processing” (MPP), may be accompanied by additional steps to improve image quality or reduce the prevalence of errors and artifacts. For example, the intensity values of pixels and voxels in the 2D images and 3D image produced at process blocks 82 and 84 may quantify an x-ray attenuation level at a given location in the subject. These attenuation levels may not be preserved when multiplying the 3D image with projected pixel values. Accordingly, more accurate indications of the attenuation levels may be restored by taking a root of the intensity value at each voxel in the time-resolved 3D image, for example, by taking the n-th root if (n−1) different sets of 2D images are used to weight the 3D image. Other processing steps can be performed before the time-resolved 3D image is delivered at process block 88.

The 2D images and 3D image produced at process blocks 82 and 84, respectively, can be produced using DSA techniques. That is, 2D images depicting the subject's vasculature can be produced by reconstructing image data acquired as a bolus of contrast passes through the ROI and subtracting out a pre-contrast, or “mask,” image acquired before the administration of contrast agent. Likewise, a 3D image of the same vascular structures can be produced by reconstructing image data acquired as contrast agent occupies the ROI and subtracting out a mask image to remove signal associated with non-vascular structures. As will be discussed below, depending on the imaging situation, the time series of 2D-DSA images and the 3D-DSA images can be produced from image data acquired using a single medical imaging system and contrast agent injection or from different sets of image data acquired separately using different medical imaging systems and contrast agent injections. In either case, the time-resolved 3D image produced by combining the DSA images depicts the subject's vascular structures with both excellent spatial and excellent temporal resolution and may thus be referred to as a 4D-DSA image. As used herein, this time-resolved 3D image may also be referred to as a 4D image, a 4D angiographic image, or a 4D DSA image The 4D-DSA images can be displayed as “pure” arterial, pure venous, or composite arterial and venous images and can be fully rotated during each state of the filling of the vasculature, thereby enabling greatly simplified interpretation of vascular dynamics. The spatial resolution of these 4D-DSA images may be on the order of 512³ pixels at about 30 frames per second. This represents an increase over traditional 3D-DSA frame rates by a factor between 150 and 600, without any significant image quality penalty being incurred. Further discussion of 4D DSA techniques may be found in U.S. Pat. No. 6,643,642, which is incorporated herein by reference in its entirety. Also, U.S. Pat. No. 8,768,031 is incorporated herein by reference, which extends the 4D DSA imaging process to use time-independent 3D rotational DSA volumes. Furthermore, US Published Patent Application US2013/0046176, which describes the use of dual-energy x-ray imaging with 4D DSA, is incorporated herein by reference.

The acquisition of contrast enhanced image data can be performed following the administration of contrast agent to the subject via either IV or IA injection. When scanning a local area, IA injections allow high image quality and temporal resolution as well as reduced contrast agent dose. However, IV injections are often more suitable for scanning larger regions where multiple IA injections at different locations and different arteries would otherwise be required. For example, there are many clinical cases where multiple 3D-DSA acquisitions, each using a different IA injection, are performed to produce separate studies that can be merged into a larger high quality vascular tree. While separate IA acquisitions may be employed for generating the time-series of 2D images used by the present invention for temporal weighting, the use of an intravenous injection for this purpose provides a mechanism for simultaneously synchronized imparting temporal information to all of the previously acquired anatomical locations present in instances when there are multiple, separate, IA 3D-DSA studies. This process reduces the likelihood of complications associated with IA contrast agent injections and improves scan efficiency.

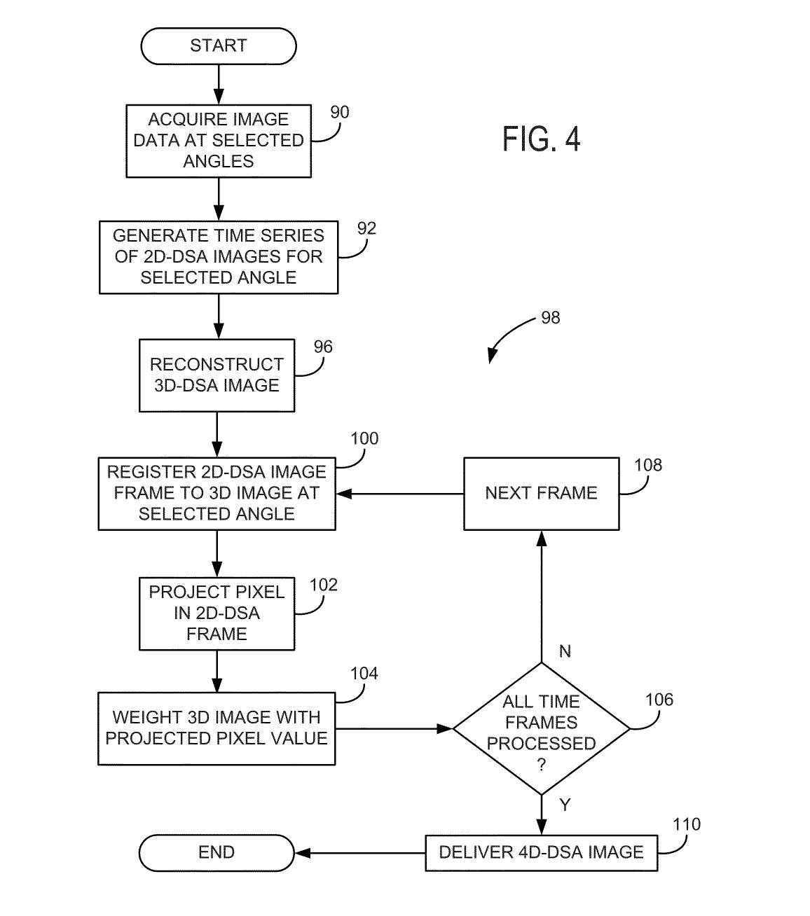

Referring to FIG. 4, a more specific implementation of the above-described process can be employed to produce a 4D-DSA image of a subject using a single-plane x-ray system in combination with a rotational x-ray system or CT system. In this case, the process begins at process block 90, when time-resolved image data from a ROI in the subject is acquired using the single-plane system following the administration of a contrast agent to the subject. Using the above-discussed DSA techniques, a time-series of 2D-DSA images at selected angles about the ROI is generated at process block 92. These 2D-DSA images depict the contrast agent passing through and enhancing arterial structures in the ROI. The 2D-DSA images are substantially free of signal from non-vascular structures, as well as signal from venous structures can be excluded due to the high temporal resolution of the 2D acquisition. A 3D-DSA image is reconstructed at process block 96 from the acquired image data. Specifically, the projections acquired at process block 90 may be log subtracted from those acquired in a non-contrast mask sweep. Typically, vascular structures in the 3D-DSA image are substantially opacified due to the use of contrast agent and the time necessary for data acquisition.

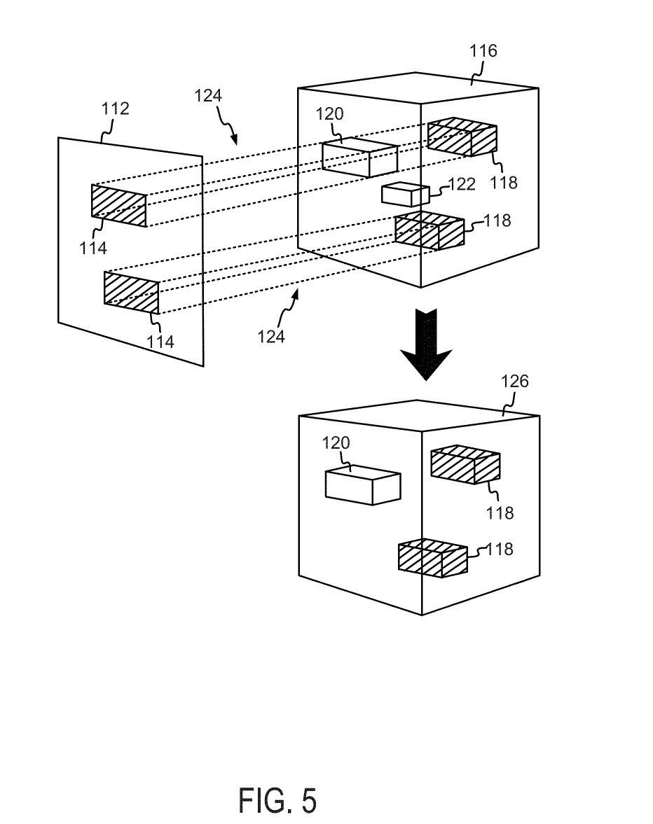

Referring now to FIGS. 4 and 5, the images produced thus far can be selectively combined with the steps indicated generally at 98 to produce a 4D-DSA image with the detailed 3D resolution of the 3D-DSA image and the temporal resolution of the time-series of 2D-DSA images. In the exemplary depiction of the selective combination process provided in FIG. 5, a single frame of the time-series of 2D-DSA images 112 includes two image regions having arterial signal 114, while the 3D-DSA image 116 includes both arterial signal 118 and venous signal 120 and 122. At process block 100 of FIG. 4, a frame of the 2D-DSA image 112 is registered to the 3D-DSA image 116 at the selected angle and, at process block 102, the values of the pixels in the 2D-DSA frame are projected along a line passing through each respective pixel in a direction perpendicular to the plane of the 2D-DSA frame. The projection of pixels with arterial signal 114 into the 3D-DSA image is indicated generally at 124. For simplicity, the projection of pixels in the 2D-DSA frame with no contrast is not shown. At process block 104 of FIG. 4, the 3D-DSA image 116 is weighted by the values projected from the 2D-DSA frame 112 to produce the 4D-DSA image 126. This may include multiplying the projected values with the voxels of the 3D image that they intersect. The weighting process results in the preservation of the arterial signal 118 and the exclusion, or “zeroing-out,” of undesired venous signal 122 in the 4D-DSA image. In addition, the intensity value of the arterial signal 114 in the 2D-DSA frame is imparted into the 3D arterial signal volume 118, thereby allowing the changes in arterial signal over time captured by the 2D-DSA images to be characterized in the 4D-DSA image. At decision block 106 of FIG. 4, if all of the frames have yet to be processed, the process moves to the next frame of the time-series of 2D-DSA images at process block 108 and repeats the selective combination process generally designated at 98. This cycle continues until, at decision block 106, it is determined that a 4D-DSA image has been generated for all relevant time frames. The 4D-DSA image can thus be delivered at process block 110.

The venous signal 120 preserved in the 4D-DSA image 126 illustrates a potential challenge when generating 4D images using only a single time-series of 2D images acquired at a single angle. That is, signal from desired structures, such as the arterial signal 114 in this example, can inadvertently be deposited in 3D voxels representing undesired structures, such as the venous region 120 in this example. The unwanted structures can thus be preserved in the 4D image as “shadow artifacts” when their signal lies along the projected values of a desired structure in a dimension inadequately characterized by the time-series of 2D images. This can result, for example, in a 4D-DSA image in which desired arterial structures are obscured by undesired venous structures for some time frames. However, this will cause a temporary anomaly in the contrast versus time course for the vein. If the time frames of the 4D-DSA image are analyzed, this anomaly can be recognized as inconsistent with the general waveform of the vein and the vein can be suppressed in the time frame where the projected arterial signal is strong. Accordingly, temporal parameters such as mean transit time (MTT) or time-to-fractional-peak can be calculated for each voxel and this information can be used to clean up shadow artifacts. To assist an operator in identifying shadow artifacts and temporal irregularities, the temporal parameters can be color-coded and superimposed on the 4D-DSA image delivered at process block 110 of FIG. 4. The temporal parameters can also be exploited to infer information related to potential perfusion abnormalities in the absence of direct perfusion information from parenchymal signal. Further still and as will be described in detail, velocity information can be used to discern arterial structures or venous structures and distinguish or discriminate between the two.

Referring again to FIG. 1A, the above described x-ray systems can be incorporated and operated according to the above-described processes to serve as the angiographic imaging system 4. To this end, referring to FIG. 1B, these systems and methods can be used to generate raw angiographic data 16 and perform 4D processing 18. As will be described additional systems can serve as the flow sensitive imaging system 2 of FIG. 1B and, thereby, acquire raw flow data and perform flow processing at process blocks 10 and 12, respectively, of FIG. 1B. Though these systems are described separately, the angiographic imaging system 4 and the flow sensitive imaging system 2 may be integrated into a single imaging modality. For example, the above-described x-ray imaging system may be integrated with an magnetic resonance imaging (MRI) system, such as will be described.

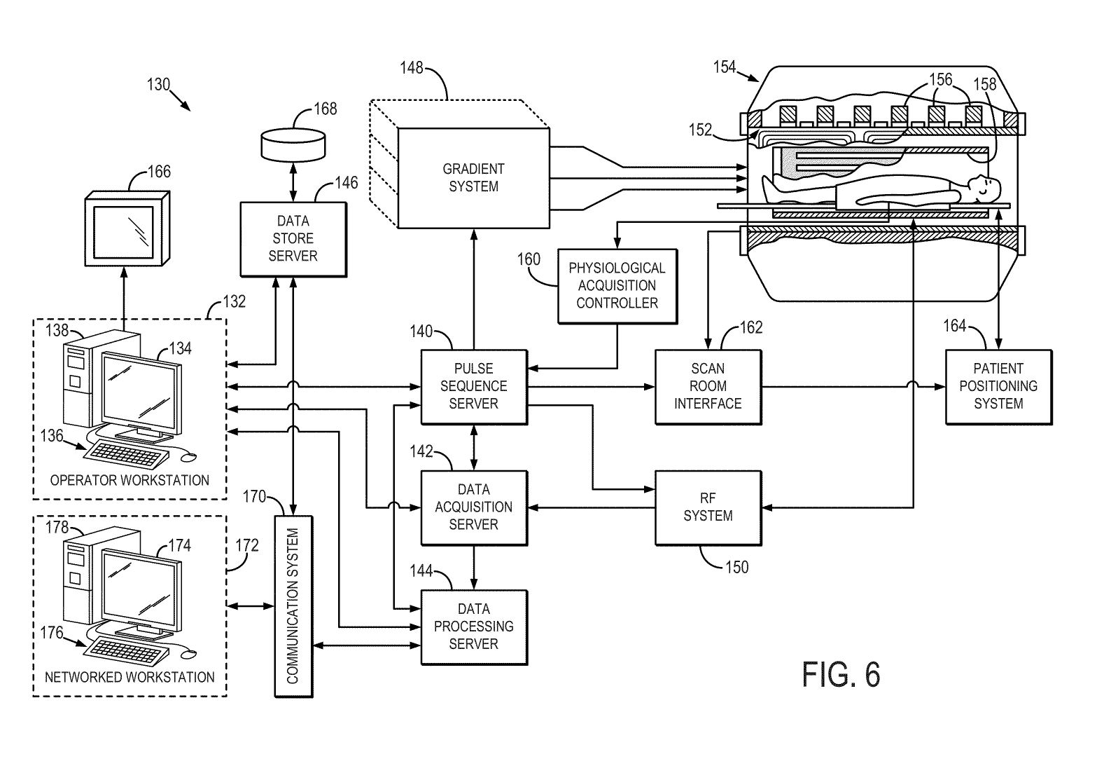

Referring particularly to FIG. 6, an example of a MRI system 130 is illustrated. The MRI system 130 includes a workstation 132 having a display 134 and a keyboard 136. The workstation 132 includes a computer system 138 that is commercially available to run a commercially-available operating system. The workstation 132 provides the operator interface that enables scan prescriptions to be entered into the MRI system 130. The workstation 132 is coupled to four actual or virtual servers: a pulse sequence server 140; a data acquisition server 142; a data processing server 144; and a data store server 146. The workstation 132 and each server 140, 142, 144, and 146 are connected to communicate with each other.

The pulse sequence server 140 functions in response to instructions downloaded from the workstation 132 to operate a gradient system 148 and a radiofrequency (RF) system 150. Gradient waveforms necessary to perform the prescribed scan are produced and applied to the gradient system 148, which excites gradient coils in an assembly 152 to produce the magnetic field gradients Gₓ, G_{y}, and G_{z} used for position encoding MR signals. The gradient coil assembly 152 forms part of a magnet assembly 154 that includes a polarizing magnet 156 and a whole-body RF coil 158 (or a head (and neck) RF coil for brain imaging).

RF excitation waveforms are applied to the RF coil 158, or a separate local coil, such as a head coil, by the RF system 150 to perform the prescribed magnetic resonance pulse sequence. Responsive MR signals detected by the RF coil 158, or a separate local coil, are received by the RF system 150, amplified, demodulated, filtered, and digitized under direction of commands produced by the pulse sequence server 140. The RF system 150 includes an RF transmitter for producing a wide variety of RF pulses used in MR pulse sequences. The RF transmitter is responsive to the scan prescription and direction from the pulse sequence server 140 to produce RF pulses of the desired frequency, phase, and pulse amplitude waveform. The generated RF pulses may be applied to the whole body RF coil 158 or to one or more local coils or coil arrays.

The RF system 150 also includes one or more RF receiver channels. Each RF receiver channel includes an RF preamplifier that amplifies the MR signal received by the coil 158 to which it is connected, and a detector that detects and digitizes the quadrature components of the received MR signal. The magnitude of the received MR signal may thus be determined at any sampled point by the square root of the sum of the squares of the I and Q components:

M=\sqrt{I²+Q²} (1);

and the phase of the received MR signal may also be determined:

$$ϕ = {tan}^{- 1} ( \frac{Q}{I} ) . \qquad ( 2 )$$

The pulse sequence server 140 also optionally receives patient data from a physiological acquisition controller 160. The physiological acquisition controller 160 receives signals from a number of different sensors connected to the patient, such as electrocardiograph (ECG) signals from electrodes, or respiratory signals from a bellows or other respiratory monitoring device. Such signals are typically used by the pulse sequence server 140 to synchronize, or “gate,” the performance of the scan with the subject's heart beat or respiration.

The pulse sequence server 140 also connects to a scan room interface circuit 162 that receives signals from various sensors associated with the condition of the patient and the magnet system. It is also through the scan room interface circuit 162 that a patient positioning system 164 receives commands to move the patient to desired positions during the scan.

The digitized MR signal samples produced by the RF system 150 are received by the data acquisition server 142. The data acquisition server 142 operates in response to instructions downloaded from the workstation 132 to receive the real-time MR data and provide buffer storage, such that no data is lost by data overrun. In some scans, the data acquisition server 142 does little more than pass the acquired MR data to the data processor server 144. However, in scans that require information derived from acquired MR data to control the further performance of the scan, the data acquisition server 142 is programmed to produce such information and convey it to the pulse sequence server 140. For example, during prescans, MR data is acquired and used to calibrate the pulse sequence performed by the pulse sequence server 140. Also, navigator signals may be acquired during a scan and used to adjust the operating parameters of the RF system 150 or the gradient system 148, or to control the view order in which k-space is sampled. In all these examples, the data acquisition server 142 acquires MR data and processes it in real-time to produce information that is used to control the scan.

The data processing server 144 receives MR data from the data acquisition server 142 and processes it in accordance with instructions downloaded from the workstation 132. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 144 are conveyed back to the workstation 132 where they may be stored. Real-time images are stored in a data base memory cache (not shown), from which they may be output to operator display 134 or a display 166 that is located near the magnet assembly 154 for use by attending physicians. Batch mode images or selected real time images are stored in a host database on disc storage 168. When such images have been reconstructed and transferred to storage, the data processing server 144 notifies the data store server 146 on the workstation 132. The workstation 132 may be used by an operator to archive the images, produce films, or send the images via a network or communication system 170 to other facilities that may include other networked workstations 142.

The communications system 140 and networked workstation 172 may represent any of the variety of local and remote computer systems that may be included within a given clinical or research facility including the system 130 or other, remote location that can communicate with the system 130. In this regard, the networked workstation 172 may be functionally and capably similar or equivalent to the operator workstation 132, despite being located remotely and communicating over the communication system 170. As such, the networked workstation 172 may have a display 174 and a keyboard 176. The networked workstation 172 includes a computer system 178 that is commercially available to run a commercially-available operating system. The networked workstation 172 may be able to provide the operator interface that enables scan prescriptions to be entered into the MRI system 130. Accordingly, as will be further described, in accordance with the present disclosure, images may be displayed and enhanced using the operator workstation 132, other networked workstations 142, or other displays 166, including systems integrated within other parts of a healthcare institution, such as an operating or emergency room and the like.

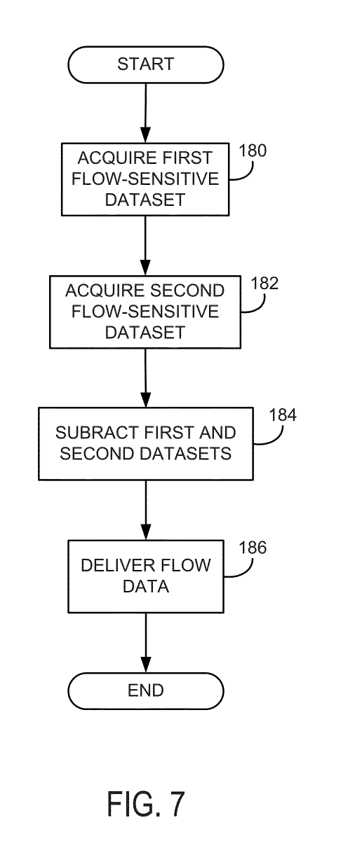

Referring to FIG. 7, a flow chart is provided that includes some non-limiting examples of steps that can be used to acquire flow data using an MRI system, such as described with respect to FIG. 6. One example of a process for acquiring flow data using an MRI system utilizes changes in phase shifts of the flowing protons in the region of interest to generate flow sensitized data and images. That is, so-called phase contrast (PC) MRI imaging can be used to acquire data from spins that are moving along the direction of a magnetic field gradient by looking for a phase shift proportional to their velocity. Specifically, referring to FIG. 6, first and second datasets are acquired at process blocks 180 and 182. Specifically, the first and second datasets are acquired with a different amounts of flow sensitivity. For example, one dataset may be flow insensitive and the other may be sensitized to flow. This may be accomplished by applying gradient pairs, which sequentially dephase and then rephase spins during the pulse sequence. Thus, for example, the first dataset acquired at process block 180 may be acquired using a “flow-compensated” pulse sequence or a pulse sequence without sensitivity to flow. The second dataset acquired at process block 182 is acquired using a pulse sequence designed to be sensitive to flow. The amount of flow sensitivity is controlled by the strength of the bipolar gradient pairs used in the pulse sequence because stationary tissue undergoes no effective phase change after the application of the two gradients, whereas the different spatial localization of flowing blood is subjected to the variation of the bipolar gradient. Accordingly, moving spins experience a phase shift. Then, at process block 184, the data from the two datasets are subtracted to yield images that illustrate the phase change, which is proportional to spatial velocity. Thus, at process block 186, the desired flow data can be delivered.

However, the process described with respect to FIG. 7 is but one general process for acquiring flow sensitized data. The above-described methods utilize phase contrast (PC) MRI imaging to acquire such data. More specifically, the above-described methods utilize a highly undersampled 3D isotropic-voxel radial projection imaging technique, often referred to as phase-contrast vastly undersampled isotropic projection reconstruction (PC VIPR). This process, as will be described, can be advantageously used to perform a 7D DSA process. However, as will be described, other imaging modalities, such as ultrasound and others, may be used to acquire flow data. Furthermore, other processes may be used to acquire flow data using an MRI system.

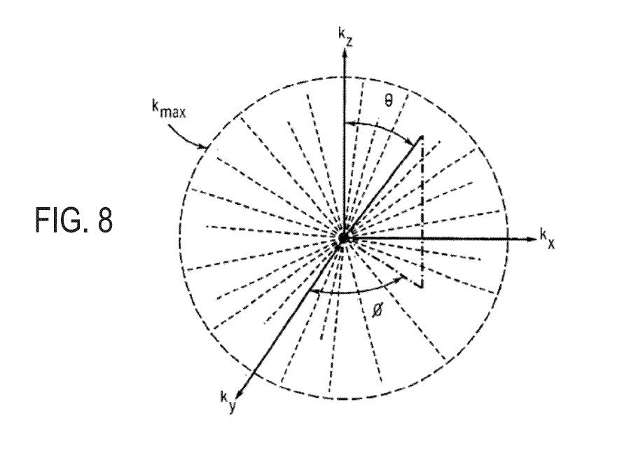

Referring to FIG. 8, data is acquired in a 3D spherical k-space coordinate system, with the readout gradient direction defined by the angle θ from the kz-axis and by the angle φ from the ky-axis. K-space sampling may be performed using a series of spaced projections with the projections going through the center of k-space. The maximum k-space radius value (kₘₐₓ) generally determines of the resulting image. The radial sample spacing (Δkᵣ) determines the diameter (D) of the full field of view (FOV) of the reconstructed image. The full FOV image may be reconstructed without artifacts if the Nyquist condition is met, Δk_{θ}, Δk_{φ}# kᵣ. If this condition is not satisfied, however, alias-free reconstruction still occurs within a reduced diameter (d) that is less than the full FOV (D). If it is assumed that the projections are acquired evenly spaced (Δk_{θ}=Δk_{φ}=Δkᵣ), then the surface area A at kₘₐₓ associated with a projection is:

$$A = Δ {k}^{2} = \frac{2 π}{{N}_{p}} {k}_{max}^{2} ; \qquad ( 3 )$$

where Nₚ is the number of acquired views, or projections. Equation (3) determines Δk, by which the diameter (d) of the reduced FOV due to the angular spacing can be related to the full FOV diameter D as follows:

$$\frac{d}{D} = \frac{2}{{N}_{R}} \sqrt{\frac{{N}_{p}}{2 π}} ; \qquad ( 4 )$$

where N_{R} is the matrix size (i.e. number of samples acquired during the signal readout) across the FOV. In the image domain, a well-constructed, reduced FOV appears centered around each object, even if the Nyquist condition is not met. However, radial streak artifacts from outside can enter the local FOV. The condition that k-space be fully sampled, or d=D, requires that the number of sampled projections be:

$${N}_{p} = \frac{π}{2} {N}_{R}^{2} . \qquad ( 5 )$$

If N_{R}=256 samples are acquired during the readout of each acquired NMR signal, for example, the number of projections Nₚ required to fully meet the Nyquist condition at the FOV diameter D is around 103,000.

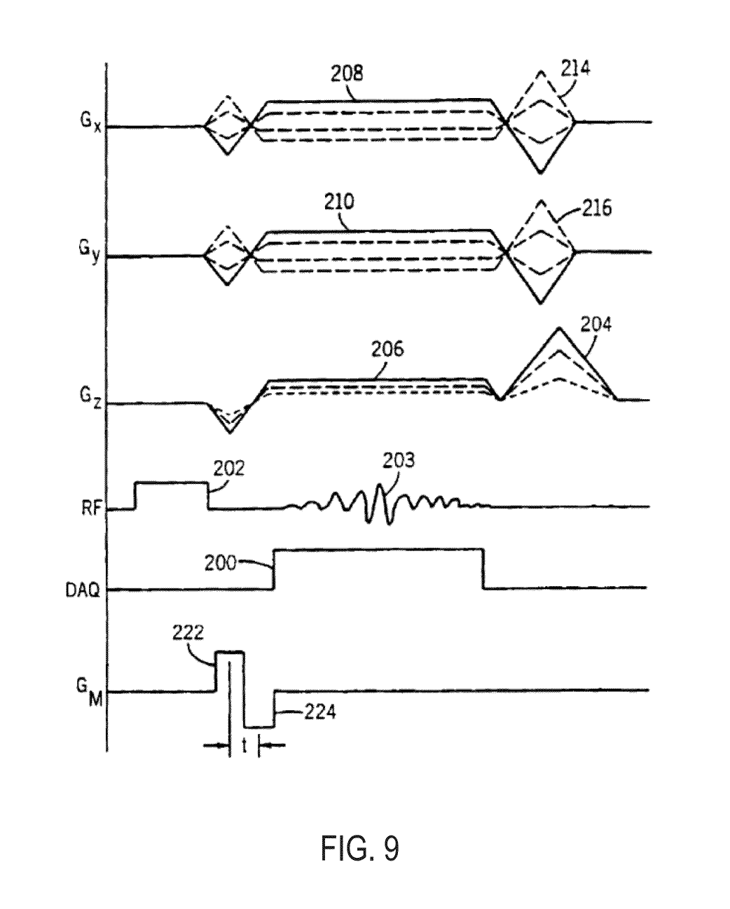

A pulse sequence used to acquire data as 3D projections is shown in FIG. 9. Either full-echo or partial-echo readouts can be performed during a data acquisition window 200. If partial echo is chosen, the bottom half of k-space (kz<0) is only partially acquired. Because of the large FOV in all directions, a non-selective 200 ms radio-frequency (RF) pulse 202 can be used to produce transverse magnetization throughout the image FOV. Relative to slab-selective excitation use in conventional spin-warp acquisitions, this method provides a more uniform flip angle across the volume, requires lower RF power, and deposits less energy into the patient.

A gradient-recalled NMR echo signal 203 is produced by spins in the excited FOV and acquired in the presence of three readout gradients 206, 208, and 210. Since a slab-select gradient is not required, the readout gradient waveforms Gₓ, G_{y}, and G_{z} may have a similar form. This symmetry is interrupted only by the need to spoil the sequence, which is accomplished by playing a dephasing gradient lobe 204. The area of the dephasing lobe 204 may be calculated to satisfy the condition:

∫₀^{T}^{_{R}}(G_{dephase}(t)+G_{read}(t))dt=n·kₘₐₓ (6);

where n is an integer n∃2. Because the G_{z} readout gradient 206 is positive on the logical z-axis, the time required for the spoiling gradient 204 is controlled by playing the dephasing lobe 204 only on G_{z}. The Gₓ and G_{y} readout gradients 208 and 210 are rewound by respective gradient pulses 212 and 214 to achieve steady state.

The readout gradient waveforms Gₓ, G_{y} and G_{z} are modulated during the scan to sample radial trajectories at different θ and φ angles. The angular spacing of θ and φ may be chosen such that a uniform distribution of k-space sample points occurs at the peripheral boundary (kₘₐₓ) of the sampled k-space sphere. Several methods of calculating the distribution are possible. One method distributes the projections by sampling the spherical surface with a spiral trajectory, with the conditions of constant path velocity and surface area coverage. This solution also has the benefit of generating a continuous sample path, which reduces gradient switching and eddy currents. For the acquisition of N total projections, the equations for the gradient amplitude as a function of projection number n are:

$$\begin{matrix}{G}_{z} = \frac{2 n - 1}{2 N} ; & ( 7 ) \\ {G}_{x} = cos ( \sqrt{2 N π} {sin}^{- 1} {G}_{z} ( n ) ) \sqrt{1 - {{G}_{z} ( n )}^{2}} ; & ( 8 ) \\ {G}_{y} = sin ( \sqrt{2 N π} {sin}^{- 1} {G}_{z} ( n ) ) \sqrt{1 - {{G}_{z} ( n )}^{2}} . & ( 9 )\end{matrix}$$

Each projection number n produces a unique project angle and when this number is indexed from 1 to N during a scan, the spherical k-space is equally sampled along all three axes.

Referring again to FIG. 9, to produce a velocity sensitive or phase contrast MRA image, each acquired projection may be motion sensitized by a bipolar motion encoding gradient G_{M}. The velocity encoding gradient G_{M} may be comprised of two gradient lobes 222 and 224 of opposite polarity. The motion encoding gradient G_{M} can be applied in any direction and it is played out after transverse magnetization is produced by the RF excitation pulse 202 and before the NMR echo signal 203 is acquired. The motion encoding gradient G_{M} imposes a phase shift to the NMR signals produced by spins moving in the direction of the gradient G_{M} and the amount of this phase shift is determined by the velocity of the moving spins and the first moment of motion encoding gradient G_{M}. The first moment (M₁) is equal to the product of the area of gradient pulse 222 or 224 and the time interval (t) between them. The first moment M₁, which is also referred to as “VENC”, is set to provide a significant phase shift, but not so large as to cause the phase to wrap around at high spin velocities.

To ensure that phase shifts in the acquired NMR signals 203 are due solely to spin motion, two acquisitions are commonly made at each projection angle and at each motion encoding gradient value M₁. One image acquisition is performed with the bipolar gradient G_{M} as shown in FIG. 8 and a second image acquisition is made with the polarity of each gradient lobe 260 and 262 reversed. The two resulting phase images are subtracted to null any phase shifts common to both acquisitions. The phase shifts caused by spin motion are also reinforced due to the reversal of motion encoding gradient polarity. An alternative technique is to acquire signals with motion encoding along each axis and then a signal with no motion encoding. The resulting reference velocity image V₀ may be subtracted from each of the motion encoded images Vₓ, V_{y} and V_{z} to null any phase shifts not caused by spin motion. With this method there is no reinforcement of the phase shifts due to motion.

As indicated above, the motion encoding gradient G_{M} can be applied in any direction. In one configuration, the motion encoding gradient G_{M} may be applied separately along each of the gradient axes, x, y and z such that an image indicative of total spin velocity can be produced. That is, an image indicative of velocity along the z axis (v_{z}) is produced by acquiring an image with the bipolar motion encoding gradient G_{M} added to the G_{z} gradient waveform shown in FIG. 8, a second velocity image Vₓ is acquired with the motion encoding gradient G_{M} added to the Gₓ gradient waveform, and a third velocity image V_{y} is acquired with the motion encoding gradient G_{M} added to the G_{y} gradient waveform. An image indicative of the total spin velocity is then produced by combining the corresponding pixel values in the three velocity images:

V_{T}=\sqrt{Vₓ²+V_{y}²+V_{z}²}. (10).

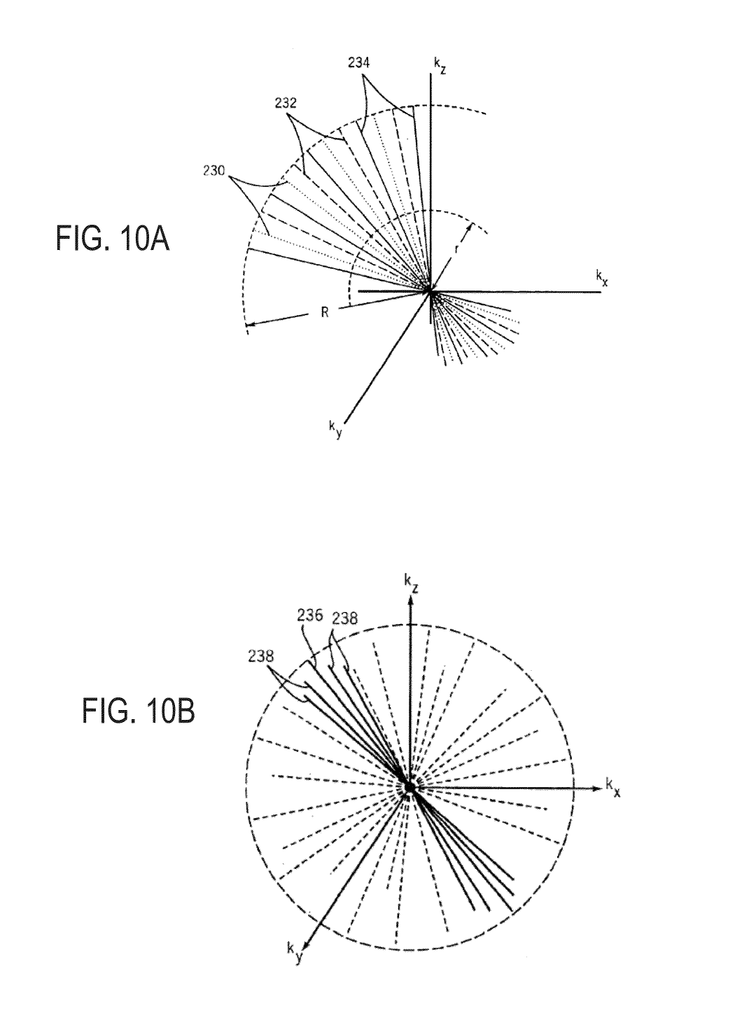

The three velocity images Vₓ, V_{y} and V_{z} are each undersampled acquisitions that may be acquired at different, interleaved projection angles. This is illustrated for one embodiment in FIG. 10A, where projection angles for the velocity image Vₓ are indicated by dotted lines 230, projection angles for image V_{y} are indicated by dashed lines 232, and projection angles for image V_{z} are indicated by lines 234. Each velocity image acquisition samples uniformly throughout the spherical k-space of radius R, but it only fully samples out to a radius r. In this embodiment both a positive and a negative motion encoding of a selected M₁ are produced along each axis of motion so that non-motion phase shifts can be subtracted out as discussed above.

In addition to the spaced projections being interleaved and uniformly spaced and acquired having different motion encoding gradient G_{M} directions (i.e., x axis, y axis and z axis), in some configurations, each of these may have a cluster of projection acquisitions thereabout having different motion encoding gradient first moments M₁. This is shown in FIG. 10B where each uniformly spaced cluster of projections includes one projection such as that indicated at 236 and a set of surrounding projections 238 having different first moments M₁. As discussed above, the different first moments M₁ are produced by varying the size or spacing of the motion encoding gradient lobes 222 and 224 in the pulse sequence of FIG. 9. All of these projections contribute to the reduction of streak artifact and at the same time produce a velocity spectrum at each reconstructed 3D image pixel. Of course, this example of the number of different velocity encoded images is not limiting and other can be achieved, for example, using different numbers of motion encoding gradients.

Therefore, the above-described system and methods can be used to produce from multiple velocity images, but that the particular number of images acquired is a matter of choice. Also, the available scan time can be used to acquire a series of velocity images depicting the subject at successive functional phases. For example, a series of 3D velocity images of the heart may be acquired and reconstructed which depict the heart at successive cardiac phases. That is, as described above, time resolved flow volumes are not required because a single flow volume may be used that shows average flow over the whole acquisition. However, it is possible to use PC VIPR acquisitions to acquire time-resolved flow data using cardiac gating. In this case, the time resolution is within the cardiac cycle, as opposed to the time during which iodine inflow occurs. Additional description of such is incorporated herein by reference to U.S. Pat. No. 6,954,067, which is incorporated herein by reference.

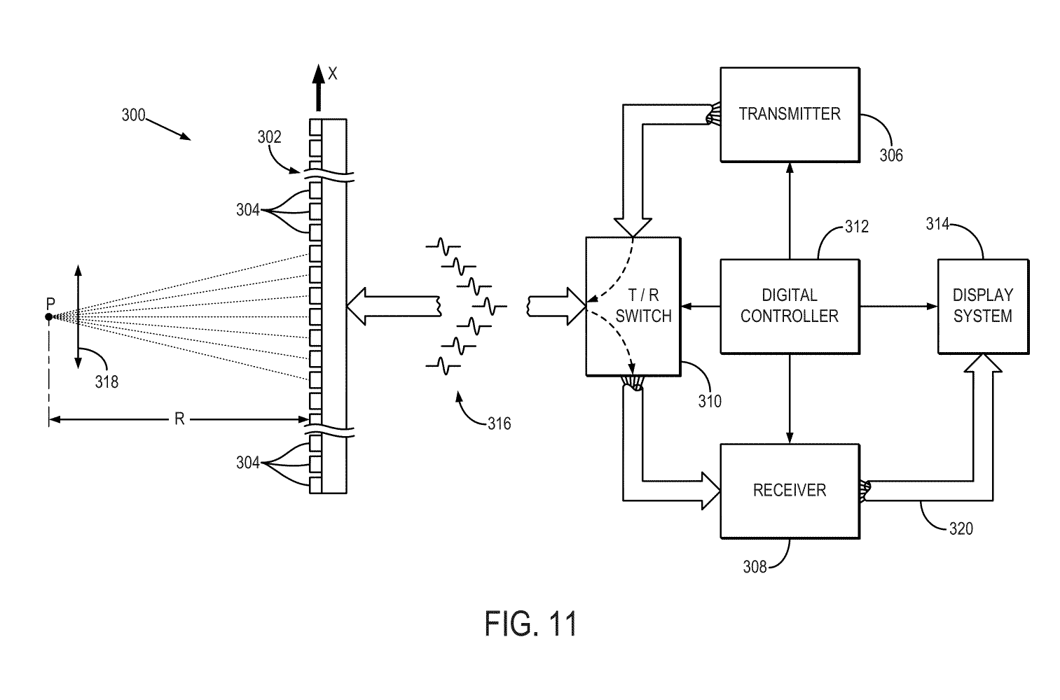

Also, the flow or velocity data can be acquired using other systems than the above-described MRI systems and methods. For example, other imaging modalities, such as ultrasound systems may be utilized to acquire the above-described flow or velocity data. Referring to FIG. 11, an example of an ultrasound imaging system 300 that may be used for implementing the present invention is illustrated. It will be appreciated, however, that other suitable ultrasound systems and imaging modalities can also be used to implement the present invention. The ultrasound imaging system 300 includes a transducer array 302 that includes a plurality of separately driven transducer elements 304. When energized by a transmitter 306, each transducer element 302 produces a burst of ultrasonic energy. The ultrasonic energy reflected back to the transducer array 302 from the object or subject under study is converted to an electrical signal by each transducer element 304 and applied separately to a receiver 308 through a set of switches 310. The transmitter 306, receiver 308, and switches 310 are operated under the control of a digital controller 312 responsive to the commands input by a human operator. A complete scan is performed by acquiring a series of echo signals in which the switches 310 are set to their transmit position, thereby directing the transmitter 306 to be turned on momentarily to energize each transducer element 304. The switches 310 are then set to their receive position and the subsequent echo signals produced by each transducer element 304 are measured and applied to the receiver 308. The separate echo signals from each transducer element 304 are combined in the receiver 308 to produce a single echo signal that is employed to produce a line in an image, for example, on a display system 314.

The transmitter 306 drives the transducer array 302 such that an ultrasonic beam is produced, and which is directed substantially perpendicular to the front surface of the transducer array 302. To focus this ultrasonic beam at a range, R, from the transducer array 302, a subgroup of the transducer elements 304 are energized to produce the ultrasonic beam and the pulsing of the inner transducer elements 304 in this subgroup are delayed relative to the outer transducer elements 304, as shown at 316. An ultrasonic beam focused at a point, P, results from the interference of the separate wavelets produced by the subgroup of transducer elements 304. The time delays determine the depth of focus, or range, R, which is typically changed during a scan when a two-dimensional image is to be performed. The same time delay pattern is used when receiving the echo signals, resulting in dynamic focusing of the echo signals received by the subgroup of transducer elements 304. In this manner, a single scan line in the image is formed.

To generate the next scan line, the subgroup of transducer elements 304 to be energized are shifted one transducer element 304 position along the length of the transducer array 302 and another scan line is acquired. As indicated at 318, the focal point, of the ultrasonic beam is thereby shifted along the length of the transducer 302 by repeatedly shifting the location of the energized subgroup of transducer elements 304.

Ultrasound systems can be used to acquire flow or velocity information. For example, Doppler ultrasound processes employ an ultrasonic beam to measure the velocity of moving reflectors, such as flowing blood cells. Blood velocity is detected by measuring the Doppler shifts in frequency imparted to ultrasound by reflection from moving blood cells. Accuracy in detecting the Doppler shift at a particular point in the bloodstream depends on defining a small sample volume at the required location and then processing the echoes to extract the Doppler shifted frequencies. In addition to targeting reflections from moving blood cells, ultrasound contrast agents, such as microbubbles, may be used.

The above-described ultrasound system 300 may be designed to perform Doppler imaging processes in a real time. The system 30 can use electronic steering and focusing of a single acoustic beam to enable small volumes to be illuminated anywhere in the field of view of the instrument, whose locations can be visually identified on a two-dimensional B-scan image. A Fourier transform processor computes the Doppler spectrum backscattered from the sampled volumes, and by averaging the spectral components the mean frequency shift can be obtained.

Typically, the calculated blood velocity is used to color code pixels in the B-scan image. Thus, as will be described, the flow data may be acquired in real time and coordinated with view changes of the 4D DSA data.

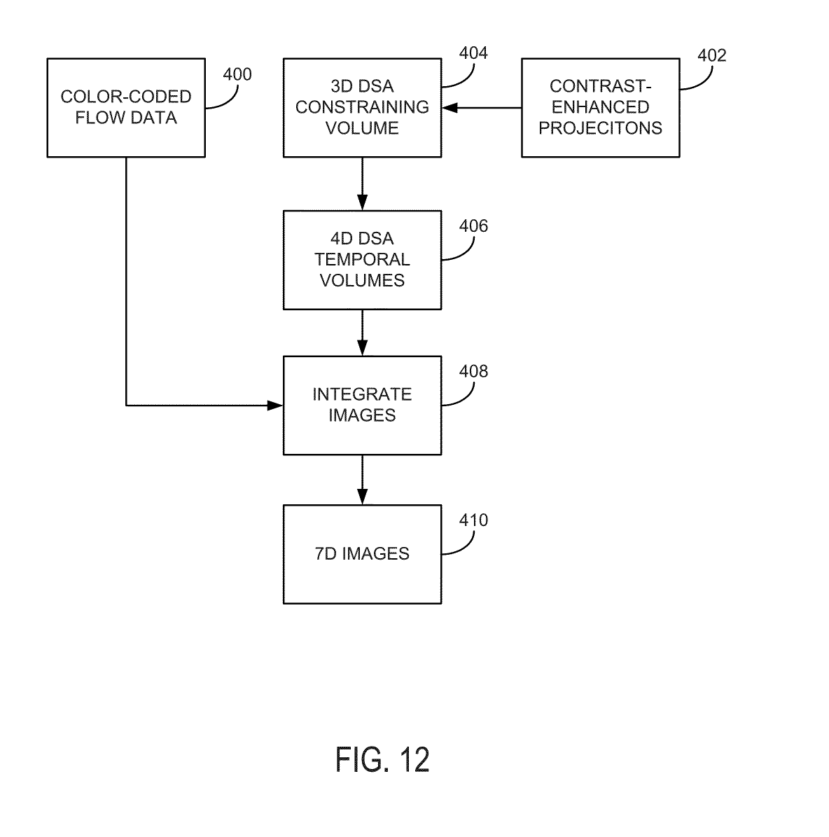

Referring now to FIG. 12, the general process described with respect to FIG. 1B is illustrated in further detail based on the intervening explanation of the above-described imaging systems and methods. In particular, at process block 400 color coded flow data is acquired. As will be described, the color-coded flow data may be acquired using any of a variety of imaging modalities, including MRI or ultrasound. To that end, it may be general phase contrast MRI data, PC VIPR MRI data, or Doppler ultrasound data, such as described above with respect to FIGS. 6-11. For example, the color-coded flow data may be PC VIPR MRI data. At process block 402, contrast-enhanced projections may be acquired using an x-ray imaging system, such as described above with respect to FIGS. 2-5. For the contrast-enhanced projections may be projections acquired using a rotational C-arm x-ray system during an iodine injection. At process block 404, the 3D DSA constraining volume is used to provide the spatial resolution and SNR for 4D DSA temporal volumes, created, for example, as described above with respect to FIGS. 3-5.

The 4D DSA temporal volume created at process block 406 and the color-coded flow data from process block 400 are then integrated at process block 408 to provide “7D images” at process block 410. As used herein “7D images” or “7D data” refer to image datasets that include spatial information in three direction (i.e., 3D volume images), including flow or velocity information in all three directions, and providing all this information over time. Thus, these 7D images or 7D data sets include three directions of spatial/anatomical information, three directions of flow or velocity information over the spatial/anatomical information, and all of this spatial/anatomical and flow or velocity information is over time, which is yet another dimension. Therefore, seven dimensions (7D) of information is provided. However, as will be described, providing such information in a clinically useful manner entails more than simply displaying anatomically registered flow data over time, such as may be created using the above-described PC VIPR MRI imaging process. Rather, the systems and methods of the present invention provide true 3D anatomical volume information, such as provided by 4D DSA processes, that include flow or velocity or, velocity derived, information.

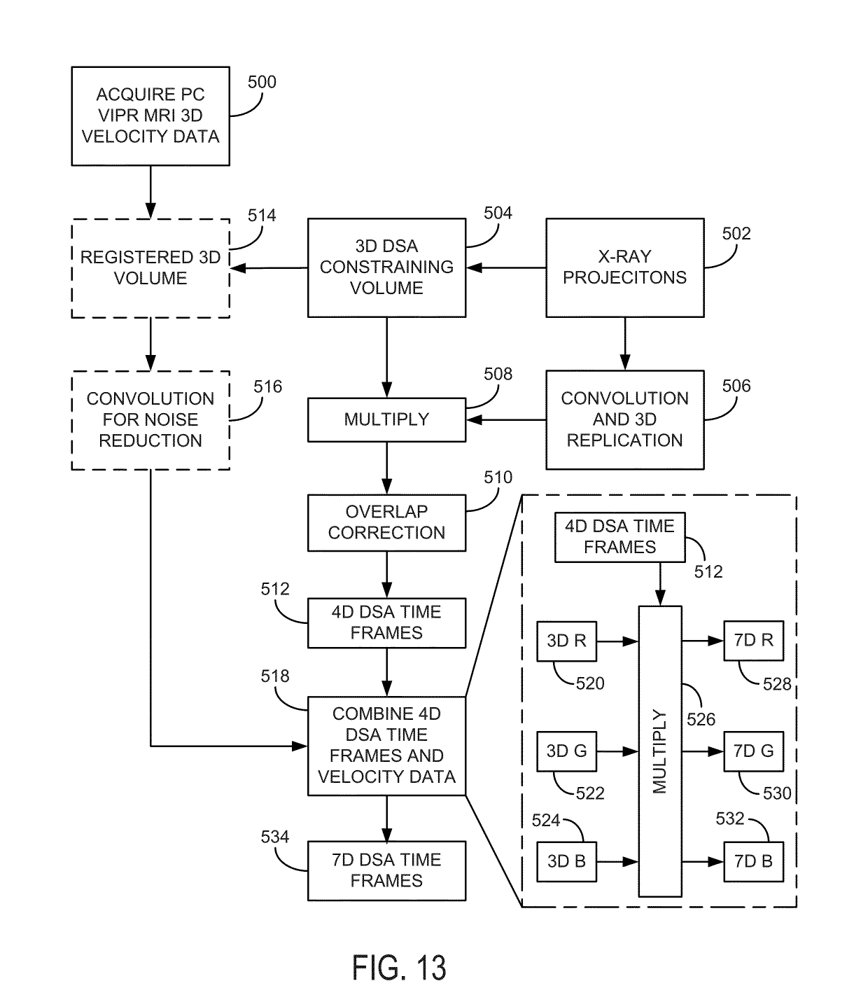

Specifically, referring to FIG. 13, the process described above with respect to FIGS. 1B and 12 are further detailed with respect to a non-limiting example. That is, FIG. 13 will be used to describe the non-limiting example of acquiring the angiographic data 16 of FIG. 1B as contrast-enhanced projections 402 of FIG. 12 using the x-ray system 30 of FIGS. 2A and 2B and of acquiring the flow data 10 of FIG. 1B as color-coded flow data 400 of FIG. 12 using the MRI system 130 of FIG. 6 in accordance with a PC VIPR imaging process, such as described with respect to FIGS. 7-10B.

The non-limiting example of FIG. 13 begins at process block 500 by acquiring color-coded flow or velocity data using the 3D PC VIPR process described above. The PC velocity data may be processed for purposes of registration or integration with 4D DSA data by multiplying a length of the three-directional velocity vector by a binarized version of PC angiogram from the 4D MR flow data, which is the complex difference volume.

This process may be performed prior to an interventional procedure, such as may be commonly guided using DSA or 4D DSA imaging. However, it is also contemplated that combined x-ray/MRI systems may be used, such that acquisition of the PC VIPR 3D velocity data at process block 500 may performed contemporaneously with an acquisition of x-ray projection data at process block 502. Regardless of whether the data acquisitions at process blocks 500 and 502 are contemporaneous or not, the x-ray projections acquired at process block 502 may then be used at process block 504 to form a 3D DSA constraining volume using the temporal information in each projection. Also, at process block 506, the x-ray projections acquired at process block 502 may be used in a convolution and 3D replication process, such as described above with respect to FIGS. 4 and 5. That is, as previously described, the angular projections acquired at process block 502 are constrained by the single 3D rotational DSA volume created at process block 504 that is made from all acquired projections, as indicated at process block 508. Thus, each angular projection is replicated through the 3D volume and convolved before voxel by voxel multiplication at process block 508. As described above with respect to FIG. 5 and the potential for overlap of undesired structures 120 with desired structures 114, overlap correction may be performed at process block 510. As such, at process block 512, a series of 4D DSA time frames is created. As described above, with respect to creating a series of 4D DSA time frames, this process may be performed in conjunction with interventional procedures, such that, for example, surgical device information may be embedded with the 4D DSA images in real time.

Referring again to the PC VIPR MRI 3D velocity data acquired at process block 500, this data may, optionally, be registered with the 3D volume information at process block 514, for example, available from the 3D DSA constraining volume. As described, this PC velocity data may have been binarized and color coded as a way to integrate the velocity information with the spatial/anatomical information provided by the 4D DSA data. As noted, the MRI and x-ray data may be acquired using a combined MRI/x-ray imaging system, in which case registration is inherently performed by the fact that the systems are combined. On the other hand, if the velocity data is acquired separately from the x-ray projections, it is advantageous to register the 3D MRI velocity data with the DSA volume at process block 514. In one configuration, registration may be aided using software, such as functional MRI of the brain (FMRIB)'s linear image registration tool (FLIRT). Additionally or alternatively, the 3D MRI velocity data may optionally convolved for noise reduction at process block 516. To the extent that that is done, the registration requirements become less precise. In either case, any registration at process block 514 may optionally include turning the velocity data and the 4D DSA data into binary representations that are subtracted to confirm proper registration. The RMS residual difference may be used as a measure of the degree of (mis)registration. If acquired separately using separate MRI or ultrasound and x-ray systems, the two data sets may include somewhat different vascular information. Thus, the result of the subtraction will often not be zero, even when the registration is optimized. However, the difference measure may serve as a metric for evaluating and improving registration. As such, process block 514 may be an iterative process that adjusts, checks, and readjusts registration until registration within a given tolerance is achieved.

At process block 518, the 3D MRI velocity data is combined with the 4D DSA time frames from process block 512. There are several options for combining information from the 4D DSA process and PC VIPR velocity data. For example, in one configuration the information from the two processes may be displayed or reported in a side-by-side fashion. Alternatively, the PC VIPR velocity data may be superimposed on the 4D DSA data using a transparent color velocity image overlaid on a gray-scale 4D DSA image. In this case, it is desirable to reduce the phase noise outside of the vessels. This can be achieved by binarizing the complex difference image and using it to multiply the velocity information. Alpha blending may also be used to create a transparent overlay upon the 4D DSA data. As yet another alternative, a color-preserving modulation process may be used to integrate the velocity information with the 4D DSA volume information.

Notably, the spatial resolution of the PC VIPR velocity data will often be lower, especially if any convolution is performed, than that of 4D DSA data. However, the change in velocity from voxel to voxel usually has a characteristically lower spatial frequency than the potentially high frequency anatomical information. The incorporation of the velocity information can proceed as in the case of the 4D DSA reconstruction where each angular projection, replicated through the 3D volume, may be convolved before voxel-by-voxel multiplication with the 4D DSA temporal volume. The extension of 4D DSA to 7D DSA is, thus, a second order application of the constrained reconstruction algorithm that produces the 4D DSA frames from the constraining image and the acquired projections.

For example, 7D DSA volumes may be reconstructed by a color preserving multiplication of the 4D DSA volumes with a time-averaged speed map created using the MRI 3D velocity data. For example, the speed map may be convolved so that the SNR and spatial resolution are provided by the 4D DSA data. The result of the second order constrained reconstruction is a dynamic display of inflowing contrast agent showing iodine concentration and arrival time as well as blood velocity.

More particularly, a color preserving modulation of the color-coded PC VIPR velocity information with the 4D DSA data can be performed such that the absolute velocity information and the iodine concentration information from the 4D DSA data are both preserved. The color preserving modulation can be achieved either by modulating the value in a hue-saturation-value (HSV) color representation or by multiplying each red-green-blue (RGB) component by the same 4D DSA data in a RGB representation. As a non-limiting example, the latter is illustrated in FIG. 13. That is, the PC VIPR MRI 3D velocity data 500 may be separated into R 520, G 522, and B 524 components that are then multiplied 526 by the 4D DSA time frames 512 to produce 7D R 528, G 530, and B 532 data. Thus, by modulating the 4D DSA time frames from process block 512 with the MRI velocity data from process block 500 using a color preserving process, the quantitative velocity related information is preserved but displayed in an integrated fashion with the high resolution 4D DSA vascular information. This second stage of the formation of the 7D images can be conceptualized as having a color flow, constraining image that gets blurred and modulated by a series of sharp 4D frames. An alternative way to conceptualize this process is that the 4D frames represent a series of sharp constraining images that are modulated by a blurred flow image.

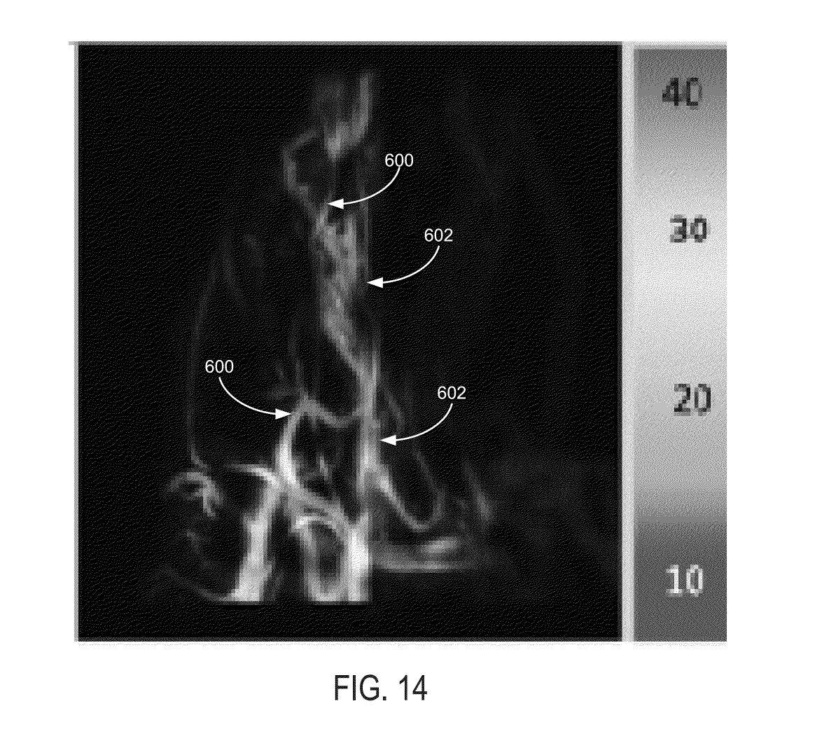

FIG. 14 provides an example of one time frame from a 7D DSA dataset. As illustrated, the underlying 4D DSA data is provided with a color-coding provided by the velocity information to show areas of higher velocity 600 and areas of lower velocity 602 in an integrated fashion. Though only a single image frame is provided for illustration purposes, the entire time-resolved 3D volume provided by the 4D DSA data is available to the clinician along with the velocity information provided by the MRI, ultrasound, or other flow-sensitive data.

Thus, a new imaging modality and imaging process is provided that combines quantitative 4D flow data with high resolution 4D DSA data to provide 7D DSA information. 4D DSA data provides fully time-resolved angiographic volumes having spatial and temporal resolution greater than that achievable with CTA or MRA alone. 4D DSA allows viewing of a contrast bolus passing through the vasculature at any time during its passage, and at any desired viewing angle. Although time-concentration curves can be extracted from a 4D data set, direct measurement of velocity or velocity-derived quantities is not possible. The present invention provides a second-order constrained reconstruction method that combines, in a single display, the high resolution anatomic detail provided by 4D DSA with the instantaneous blood flow information (velocity and velocity-derived quantities) provided by 4D flow MRI. Such velocity-derived quantities may include pressure gradient information, wall shear stress, flow streamline information, and the like.

Such comprehensive information can provide a means to examine complex structures from arbitrary angles with a temporal resolution of 30 volumes per second while also providing physiological velocity-derived information. This provides new methods for treatment planning for arterio-venous malformations with complicated filling and draining patterns, fistulas, and aneurysms.

The present invention has been described in terms of one or more preferred embodiments, and it should be appreciated that many equivalents, alternatives, variations, and modifications, aside from those expressly stated, are possible and within the scope of the invention.

1. A system for generating time resolved series of angiographic volume data having velocity or velocity-derived information integrated therewith, the system comprising:
an image processing system configured to receive angiographic volume data and flow sensitive imaging data and process the angiographic volume data and flow sensitive imaging data to generate a combined dataset; and
a display configured to display the combined dataset as angiographic volumes having associated time-resolved, color-coded flow information.

2. The system of claim 1 wherein the flow sensitive imaging data includes velocity encoded MRI data.

3. The system of claim 1 wherein the flow sensitive imaging data includes 3D Doppler ultrasound data.

4. The system of claim 1 wherein the angiographic volume data includes x-ray projection data.

5. The system of claim 1 wherein the x-ray projection data includes four-dimensional (4D) digital subtraction angiography data.

6. The system of claim 1 wherein the image processing system is configured to co-register the angiographic volume data and flow sensitive imaging data in 3D space.

7. The system of claim 6 wherein the image processing system is further configured to perform co-registration using the angiographic volume data as a constraining volume.

8. The system of claim 1 wherein the image processing system is further configured to generate the combined dataset by multiplying the angiographic volume data and the flow sensitive imaging data.

9. The system of claim 1 wherein the flow sensitive imaging data includes a series of flow-sensitive 3D volumes.

10. The system of claim 9 wherein the series flow-sensitive 3D volumes includes a measure of velocity in one of three (x,y,z) directions or net velocity.

11. The system of claim 1 wherein the time-resolved, color-coded flow information includes at least one of velocity measurements, pressure gradient information, wall shear stress, or flow streamline information.

12. The system of claim 1 wherein the image processing system is configured to generate the combined dataset by multiplying components of the flow-sensitive imaging data by the angiographic volume data without changing spatial information of the angiographic volume data or flow information of the flow-sensitive imaging data.

13. The method of claim 1 wherein the image processing system is configured to generate the combined dataset by spatially convolving the flow-sensitive volume data to increase a signal to noise ratio (SNR).

14. A method for generating time resolved series of angiographic volume data having velocity or velocity-derived information integrated therewith, the method comprising:
generating a series of 3D time-resolved vascular volumes from time resolved x-ray projection data;
generating a series of flow-sensitive 3D volumes from one of magnetic resonance imaging data or ultrasound data; and
integrating the series of 3D time-resolved vascular volumes and the series flow-sensitive 3D volumes to generate a series of time-resolved vascular volumes that have voxel intensities or color coding defined by at least one of iodine concentration derived from the time resolved x-ray projection data or velocity information derived from the one of magnetic resonance imaging data or ultrasound data.

15. The method of claim 14 wherein the integrating includes multiplying the 3D time-resolved vascular volumes by the series flow-sensitive 3D volumes.

16. The method of claim 14 wherein the series flow-sensitive 3D volumes includes a measure of velocity in one of three (x,y,z) directions or net velocity.

17. The method of claim 14 wherein the velocity information includes at least one of velocity measurements, pressure gradient information, wall shear stress, or flow streamline information.

18. The method of claim 14 wherein generating the series of 3D time-resolved vascular volumes includes operating an x-ray imaging system to derive four-dimensional (4D) digital subtraction angiography (DSA) imaging data.

19. The method of claim 18 further comprising using an overall time-independent 3D rotational volume to derive the 4D DSA imaging data.

20. The method of claim 14 wherein the time resolved x-ray projection data include dual-energy x-ray projection data.

21. The method of claim 14 wherein the integrating includes multiplying components of the series of flow-sensitive 3D volumes by the series of 3D time-resolved vascular volumes without changing spatial information of the 3D time-resolved vascular volumes or flow information of the series of flow-sensitive 3D time-resolved volumes.

22. The method of claim 14 wherein the integrating includes spatially convolving the series of flow-sensitive 3D volumes to increase a signal to noise ratio (SNR).

23. The method of claim 14 wherein the series flow-sensitive 3D volumes include time-resolved flow-sensitive 3D volumes.

24. A system for generating time resolved series of angiographic volume data having velocity or velocity-derived information integrated therewith, the system comprising:
a source of x-ray data of a subject having received a dose of a contrast agent;
a source of at least one of magnetic resonance imaging data or ultrasound data of the subject;
a processing system having access to the source of x-ray data and the source of at least one of magnetic resonance imaging data or ultrasound data and configured to:
generate a time-series of two-dimensional images from the x-ray data, each of the two-dimensional images corresponding to a different time in the time period and a different angle relative to the subject, wherein each of the two-dimensional images comprises pixel intensity information;
generate a three-dimensional image without temporal resolution from the x-ray data;
determine, for each of a plurality of the two-dimensional images, voxel weightings in the three-dimensional image without temporal resolution by multiplying the voxels with the pixel intensity information of a two-dimensional image in the plurality;
producing a time-resolved three-dimensional image of the subject by selectively combining the three-dimensional image without temporal resolution and the time-series of two-dimensional images, the voxel weightings being used to nullify one or more voxels from the three-dimensional image without temporal resolution to produce the time-resolved three-dimensional image;
produce a series of velocity images of the subject, wherein each of the velocity images comprises pixel color information weighted based on flow information associated derived from the at least one of magnetic resonance imaging data or ultrasound data; and
combine the series of velocity images of the subject with the time-resolved three-dimensional image of the subject to generate a series of time-resolved vascular volumes that have voxel intensities or color coding defined by the flow information.

25. The system of claim 24 wherein the processing system is configured to multiply the velocity images of the subject and the time-resolved three-dimensional image of the subject to perform the combining.

26. The system of claim 25 wherein the processing system is configured to binarize the series of velocity images of the subject and multiply a three-directional velocity vector of the binarized series of velocity images and the time-resolved three-dimensional image to perform the combining.

27. The system of claim 25 wherein the processing system is configured to multiply each of a red, green, and blue component of pixel color information weighted based on flow information and the time-resolved three-dimensional image to perform the combining.
